(19) United States
(12) Patent Application Publication
(10) Pub. No.: US 2023/0017308 A1
(43) Pub. Date: Jan. 19, 2023

(54) METHOD AND APPARATUS FOR VIRTUAL HEALTHCARE ASSISTANT

(71) Applicant: AT&T Intellectual Property I, L.P., Atlanta, GA (US)

(72) Inventor: Wei Wang, Harrison, NJ (US)

(73) Assignee: AT&T Intellectual Property I, L.P., Atlanta, GA (US)

(21) Appl. No.: 17/375,477

(22) Filed: Jul. 14, 2021

(51) Int. Cl. G16H 40/67; G16H 40/20; G16H 20/13; G16H 80/00; G06K 19/077; G06K 19/07; G16H 10/60; G16H 50/20; H04W 4/024; H04L 29/06; G08B 21/24; A61J 7/04

(52) U.S. Cl. CPC G16H 40/67; G16H 40/20; G16H 20/13; G16H 80/00; G06K 19/0776; G06K 19/0723; G16H 10/60; G16H 50/20; H04W 4/024; H04L 65/1069; G08B 21/24; A61J 7/0481; A61J 7/0436; A61J 2200/30; A61J 2205/60

(57) ABSTRACT

Aspects of the subject disclosure may include, for example, receiving, by a processing system including a processor of an end user device of a user, medication information indicating a reminder to take a medication, where the medication information is received over a wireless network from a virtual healthcare assistant operating on a customer device located at a premises, and where the medication information indicates an identification of a wireless transceiver of a medicine container that houses the medication to be dispensed. The end user device determines a user position and determines a container position of the medicine container. The end user device presents navigation information that facilitates movement by the user towards the medicine container. In other embodiments, a display device can be from among a group of display devices according to a position of the user in the premises; and a video conference can be established with the communication device of the healthcare provider that is presented at the display device of the user. Other embodiments are disclosed.

### FIELD OF THE DISCLOSURE

The subject disclosure relates to a method and apparatus for a virtual healthcare assistant.

### BACKGROUND

It is believed that the number of healthcare workers needs to increase dramatically to meet demand in the coming years. Forecasts have identified a significant upcoming shortage of home health aides and nurse practitioners that help patients or the elderly carrying out at least some of their daily activities, including at home. These daily activities can be quite critical such as daily medicine to be taken and scheduling/attending a doctor's appointment.

### BRIEF DESCRIPTION OF THE DRAWINGS

Reference will now be made to the accompanying drawings, which are not necessarily drawn to scale, and wherein:

FIG. 1 is a block diagram illustrating an exemplary, non-limiting embodiment of a communications network in accordance with various aspects described herein.

FIGS. 2A-2D are block diagrams illustrating example, non-limiting embodiments of systems that provide a virtual healthcare assistant functioning within the communication network of FIG. 1 in accordance with various aspects described herein.

FIG. 2E depicts an illustrative embodiment of a method in accordance with various aspects described herein.

FIG. 3 is a block diagram illustrating an example, non-limiting embodiment of a virtualized communication network in accordance with various aspects described herein.

FIG. 4 is a block diagram of an example, non-limiting embodiment of a computing environment in accordance with various aspects described herein.

FIG. 5 is a block diagram of an example, non-limiting embodiment of a mobile network platform in accordance with various aspects described herein.

FIG. 6 is a block diagram of an example, non-limiting embodiment of a communication device in accordance with various aspects described herein.

### DETAILED DESCRIPTION

The subject disclosure describes, among other things, illustrative embodiments for a virtual healthcare assistant to facilitate providing healthcare to a user, such as at a premises. The healthcare can be of various types including dispensing medication, providing doctor's appointments, coordinating medical events, recording events that have occurred, and so forth. Other embodiments are described in the subject disclosure.

One or more embodiments can implement communication technologies to facilitate healthcare-related activities so as to make it easier for the patients or elderly and to reduce the workload of healthcare workers. One or more embodiments can implement a smart or virtual healthcare assistant at a premises or home to perform various functionality including organizing a day for a user and/or assisting the user with healthcare-related activities such as through use of smart medicine containers. There is pressure on both human resources and facilities to meet the high demand of healthcare in the coming years. One or more of the exemplary embodiments facilitate and increase home-based health care which can prevent exhausting crowded public facilities such as nursing houses and hospitals. The exemplary embodiments can improve family life, safety for the home occupants, and ease of handling various, possibly complex, situations associated with healthcare.

In one or more embodiments, a virtual healthcare assistant can be integrated with an existing smart home assistant (e.g., various types of devices such as routers, smart home hardware/software, smart management devices, or any device that can be an integrator and can have connection to networks) at a premises. In other embodiments, the virtual healthcare assistant can be a stand-alone device or executed by one or more other devices, such as fixed or mobile devices located at the premises including in a centralized or distributed fashion. In one or more embodiments, the virtual healthcare assistant can notify a user or someone that assists the user of scheduled events, locations of events, locations of medical supplies, and so forth.

In one or more embodiments, a registration function can be performed. For example, in order to manage daily activities for a person, the agenda of the person can be stored or otherwise obtained by the virtual healthcare assistant. For example, the registration can be pushed by communication devices of multiple parties, such as from a doctor's office or by a physical therapist. The virtual healthcare assistant can order the events by time.

In one or more embodiments, on-boarding of medicine containers can be performed. For example, when a container, such as a pill bottle, is brought into the house, in order for the virtual healthcare assistant to know which pill bottle corresponds to which medication, the pill bottle can be equipped with a chip or other circuit that enables communications and/or processing. An identifier can be stored in the chip which may also include the medication name. In one embodiment, the chip of the pill bottle is not required to initiate registration with the virtual healthcare assistant, but the virtual healthcare assistant can enable a user to search for a particular ID such as at or near the time of an event (e.g., a scheduled time for taking medication).

In one or more embodiments, utilizing localization technology, such as Ultra-wide band communications, a pill bottle chip can also be used for user navigation and locating of the pill bottle such as with an accuracy of at least a centimeter-level. In one embodiment, the pill bottle can also have a cap sensor or other sensor that tracks or monitors medication being dispensed, such as a dispensing cap that counts the number of pills dispensed from the pill bottle (and/or the number of pills provisioned into the pill bottle) in order to track dosage. In one embodiment, the medicine container can be equipped with a weight sensor, such as for liquid medication. The medication that is dispensed can be prescription medication and/or non-prescription medication, where the medication is of various types (including vitamins, supplements, over-the-counter medication, and so forth) and various forms (e.g., pills, capsules, liquid, powder, and so forth). In one or more embodiments, the virtual healthcare assistant can monitor and facilitate locating of other medical supplies or medical equipment such as bandages, aerosol sprays, and so forth. In one or more embodiments, the virtual healthcare assistant can monitor, reminder, guide and/or facilitate other therapeutic activities for the user such as a reminder to go to the garden for a 15 minute walk.

In one or more embodiments, event management can be performed by the virtual healthcare assistant. For example, at 8:00 AM, a user at home may be scheduled to take medicine X. The virtual healthcare assistant can push a notification to the user. For instance, the user can have a wearable communication device, such as a smart watch or a smart healthcare wristband, to receive such a notification, or it can be received via another device such as a smart phone. Upon receipt of the notification, the user would start looking for that pill bottle with a specific ID in the house. Navigation instructions can be performed by the virtual healthcare assistant to guide the user towards the correct medicine container, such as through use of Ultra-Wideband messages being sent between the medicine container, the virtual healthcare assistant and/or the user wearable device (or smart phone). In one or more embodiments, the medicine container or pill bottle can monitor or otherwise count a number of pills dispensed and report back to the virtual healthcare assistant and/or smart home manager/assistant, including time of dispensing, amount of dispensing and/or amount of medication that is left to be dispensed.

In one or more embodiments, appointment management can be performed by the virtual healthcare assistant. For example, upon an appointment event, such as an online doctor appointment, the virtual healthcare assistant can ping or otherwise notify the wearable device of the user (e.g., smart watch or wrist band on the user) and determine the location of the user (e.g., via localization messaging techniques between the virtual healthcare assistant and the wearable device). The nearest or otherwise preferred display device with a camera can be identified, activated, and connected to a video conference with equipment of the doctor's office, such as according to a user confirmation of initiating the video conference.

In one or more embodiments, refill management for medical supplies including medication can be performed by the virtual healthcare assistant. For example, when a medicine container reaches a threshold or is otherwise close to being depleted of medication, the medicine container (e.g., via a chip connected with the medicine container) can wirelessly report back to the virtual healthcare assistant for auto-refill of the medication and/or message coordination with the equipment of a doctor's office can be employed, such as to update medical records, obtain a prescription, and so forth.

In one or more embodiments, other healthcare management can be performed by the virtual healthcare assistant. For example, other activities including healthcare related activities can also be coordinated by the virtual healthcare assistant and/or smart home assistant including scheduling the user's activities and providing the user with reminders for the activities, where localization sensors are in place with respect to the user and any equipment associated with the activities. In one embodiment, location sensors can navigate the user to objects as well as providing (or notifying) close-by objects to the user.

One or more aspects of the subject disclosure include a device comprising: a processing system including a processor; and a memory that stores executable instructions that, when executed by the processing system, facilitate performance of operations. The device can determine a scheduled appointment at a future time for a user that is a resident of a premises in which the device is located. The device can receive, over a wireless network operating in the premises, dispensing data from a wireless transmitter of a medicine container, the dispensing data indicating a time of dispensing and an identification of medication that was dispensed. The device can transmit the dispensing data to a communication device associated with a healthcare provider. The device can determine, within a time period of the scheduled appointment, a position of the user in the premises based on communications associated with a wearable end user device that is worn by the user. The device can select a display device from among a group of display devices according to the position of the user in the premises and can facilitate establishing a video conference with the communication device of the healthcare provider that is presented at the display device of the user.

One or more aspects of the subject disclosure include a method comprising receiving, by a processing system including a processor of an end user device of a user, medication information indicating a reminder to take a medication, where the medication information is received over a wireless network from a virtual healthcare assistant operating on a customer device located at a premises where a user is a resident, and where the medication information indicating an identification of a wireless transceiver of a medicine container that houses the medication to be dispensed. The method includes determining, by the processing system, a user position of the user that is remote from the premises. The method includes determining, by the processing system, a container position of the medicine container that is remote from the premises based on communications with the wireless transceiver of the medicine container. The method includes presenting, by the processing system at a display of the end user device, navigation information that facilitates movement by the user towards the medicine container.

One or more aspects of the subject disclosure include a non-transitory machine-readable medium, comprising executable instructions that, when executed by a processing system including a processor operating on a customer device located at a premises where a user is a resident, facilitate performance of operations to provide a virtual healthcare assistant. The operations include obtaining registration information for a group of medicine containers that house different medications. The operations include transmitting, to a wearable end user device of the user according to a medication schedule, medication information indicating a reminder to take a medication. The operations include selecting a medicine container that houses the medication from among the group of medicine containers according to the medication schedule and the registration information. The operations include transmitting instructions to the wearable end user device that causes the wearable end user device to present navigation information that directs movement of the user towards the medicine container, wherein the navigation instructions are based on localization messaging associated with the wearable end user device and an RFID label of the medicine container.

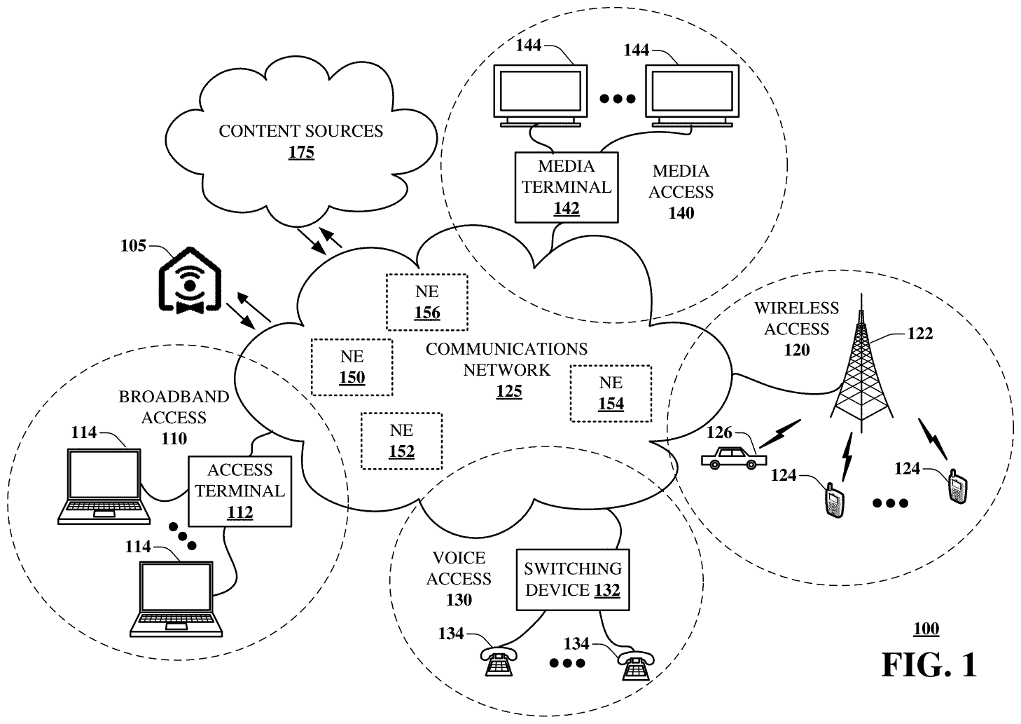

Referring now to FIG. 1, a block diagram is shown illustrating an example, non-limiting embodiment of a system 100 in accordance with various aspects described herein. For example, system 100 can facilitate in whole or in part determining a scheduled appointment at a future time for a user that is a resident of a premises in which the device is located; receiving, over a wireless network operating in the premises, dispensing data from a wireless transmitter of a medicine container, the dispensing data indicating a time of dispensing and an identification of medication that was dispensed; transmitting the dispensing data to a communication device associated with a healthcare provider; determining, within a time period of the scheduled appointment, a position of the user in the premises based on communications with a wearable end user device that is worn by the user; selecting a display device from among a group of display devices according to the position of the user in the premises; and facilitating establishing a video conference with the communication device of the healthcare provider that is presented at the display device of the user. System 100 can include a virtual healthcare assistant operating on a customer device 105 such as located at a premises where a user is a resident. The device 105 can communicate with medicine containers (not shown) including transmitting and/or receiving medication information, location data, and so forth. In one embodiment, the device 105 can include an RFID generator that provisions RFID labels that are to be applied to or are already applied to (or connected with) medicine containers where the provisioning provides information that facilitates the embodiments described herein including locating particular medicine containers having particular medications, wirelessly communicating with a virtual healthcare assistant operating on a customer device located at a premises where a user is a resident, determining dispensing of medication from the medicine container, determining a count of dispensed medication from the medicine container, or combination thereof. The provisioned information can be various types of information including radio IDs, medication IDs, executable code, scheduling information, and so forth.

In particular, a communications network 125 is presented for providing broadband access 110 to a plurality of data terminals 114 via access terminal 112, wireless access 120 to a plurality of mobile devices 124 and vehicle 126 via base station or access point 122, voice access 130 to a plurality of telephony devices 134, via switching device 132 and/or media access 140 to a plurality of audio/video display devices 144 via media terminal 142. In addition, communication network 125 is coupled to one or more content sources 175 of audio, video, graphics, text and/or other media. While broadband access 110, wireless access 120, voice access 130 and media access 140 are shown separately, one or more of these forms of access can be combined to provide multiple access services to a single client device (e.g., mobile devices 124 can receive media content via media terminal 142, data terminal 114 can be provided voice access via switching device 132, and so on).

The communications network 125 includes a plurality of network elements (NE) 150, 152, 154, 156, etc. for facilitating the broadband access 110, wireless access 120, voice access 130, media access 140 and/or the distribution of content from content sources 175. The communications network 125 can include a circuit switched or packet switched network, a voice over Internet protocol (VoIP) network, Internet protocol (IP) network, a cable network, a passive or active optical network, a 4G, 5G, or higher generation wireless access network, WIMAX network, UltraWideband (UWB) network, personal area network or other wireless access network, a broadcast satellite network and/or other communications network.

In various embodiments, the access terminal 112 can include a digital subscriber line access multiplexer (DSLAM), cable modem termination system (CMTS), optical line terminal (OLT) and/or other access terminal. The data terminals 114 can include personal computers, laptop computers, netbook computers, tablets or other computing devices along with digital subscriber line (DSL) modems, data over coax service interface specification (DOCSIS) modems or other cable modems, a wireless modem such as a 4G, 5G, or higher generation modem, an optical modem and/or other access devices.

In various embodiments, the base station or access point 122 can include a 4G, 5G, or higher generation base station, an access point that operates via an 802.11 standard such as 802.11n, 802.11ac or other wireless access terminal. The mobile devices 124 can include mobile phones, e-readers, tablets, phablets, wireless modems, and/or other mobile computing devices.

In various embodiments, the switching device 132 can include a private branch exchange or central office switch, a media services gateway, VoIP gateway or other gateway device and/or other switching device. The telephony devices 134 can include traditional telephones (with or without a terminal adapter), VoIP telephones and/or other telephony devices.

In various embodiments, the media terminal 142 can include a cable head-end or other TV head-end, a satellite receiver, gateway or other media terminal 142. The display devices 144 can include televisions with or without a set top box, personal computers and/or other display devices.

In various embodiments, the content sources 175 include broadcast television and radio sources, video on demand platforms and streaming video and audio services platforms, one or more content data networks, data servers, web servers and other content servers, and/or other sources of media.

In various embodiments, the communications network 125 can include wired, optical and/or wireless links and the network elements 150, 152, 154, 156, etc. can include service switching points, signal transfer points, service control points, network gateways, media distribution hubs, servers, firewalls, routers, edge devices, switches and other network nodes for routing and controlling communications traffic over wired, optical and wireless links as part of the Internet and other public networks as well as one or more private networks, for managing subscriber access, for billing and network management and for supporting other network functions.

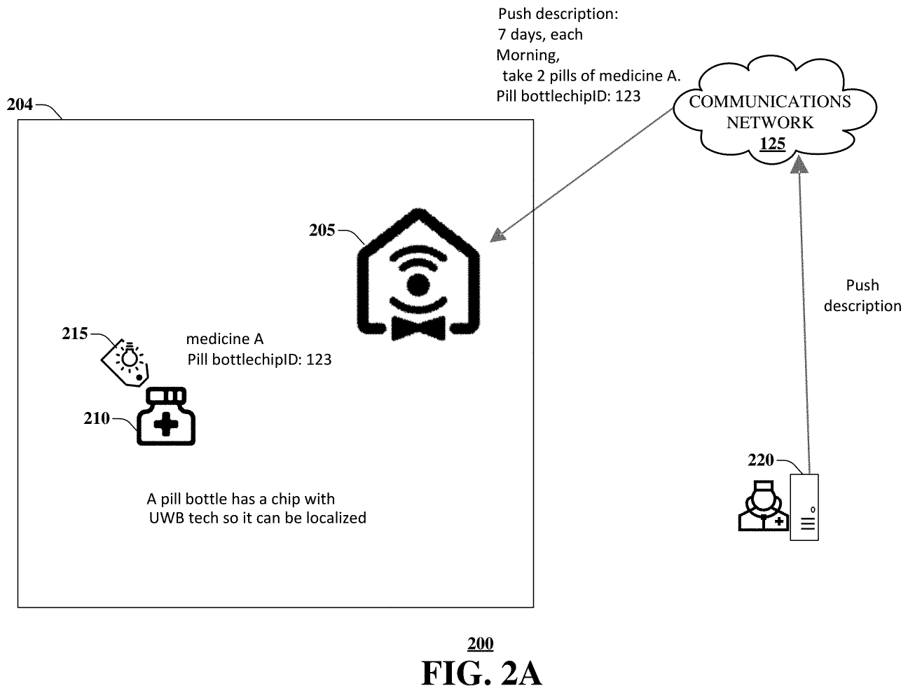

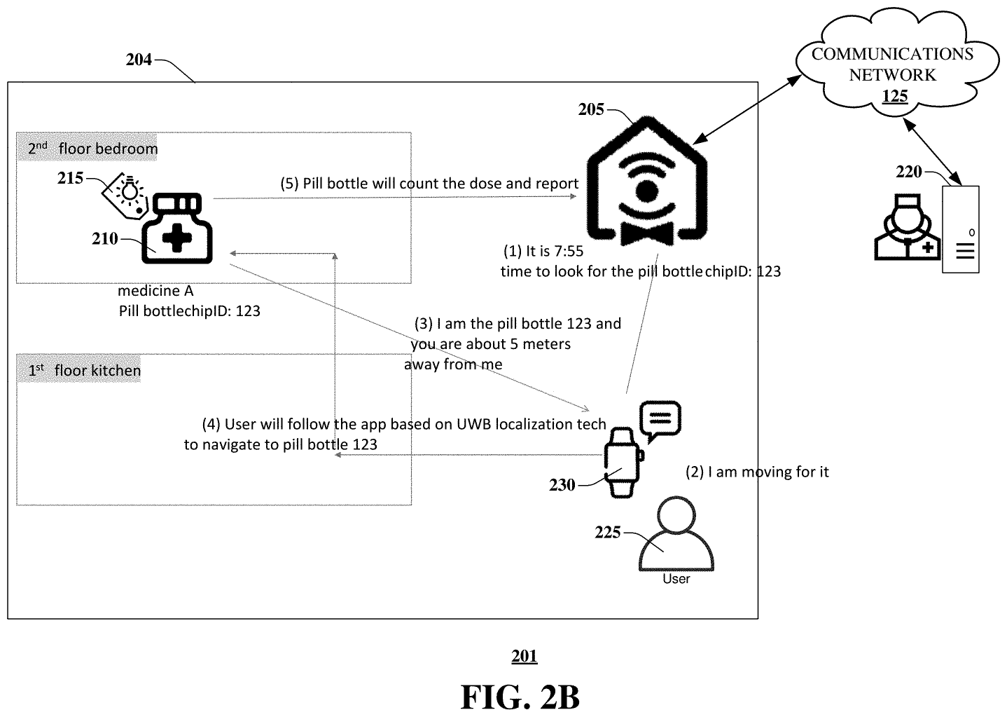

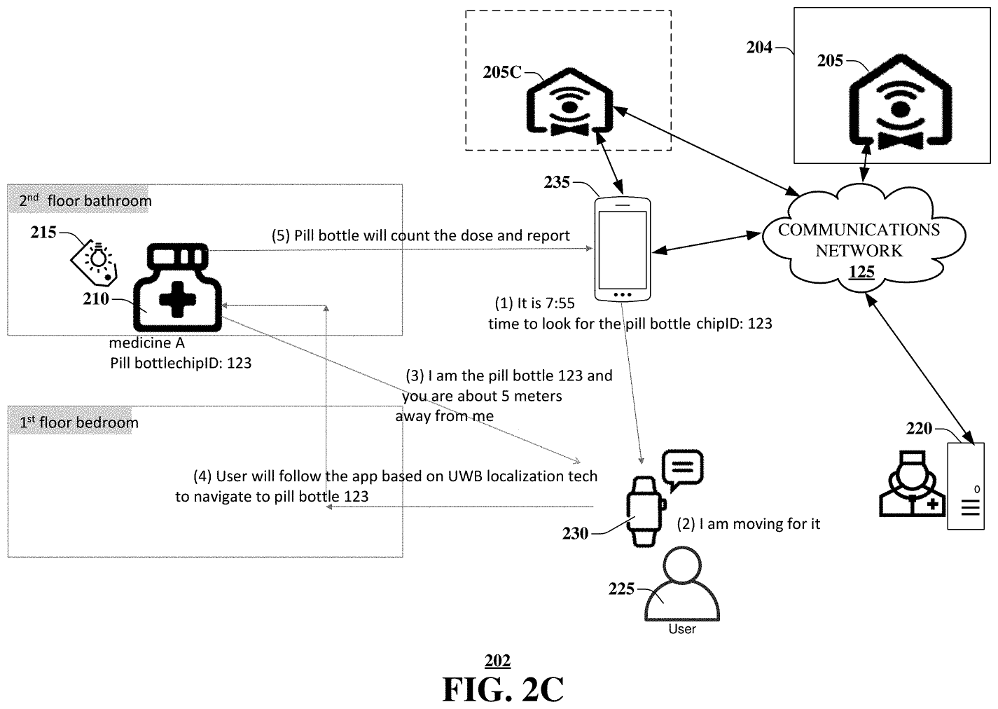

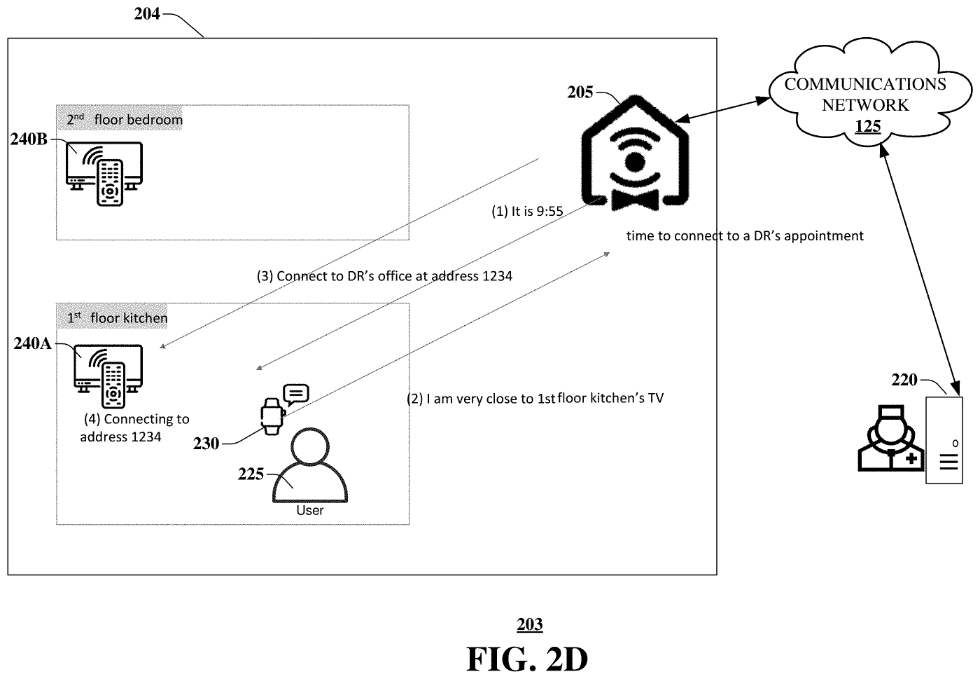

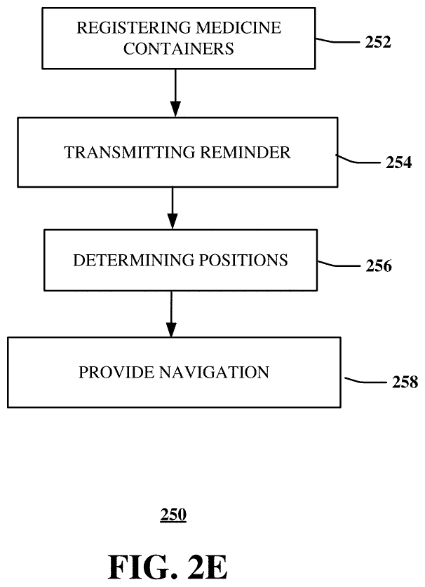

FIG. 2A is a block diagram illustrating an example, non-limiting embodiment of a system 200 functioning within the communication network of FIG. 1 in accordance with various aspects described herein. System 200 illustrates an example of medication dispensing management including registration of medicine containers (and/or other medical supplies and medical devices) through use of a customer device 205 (e.g., operating as or otherwise executing a virtual healthcare assistant which can be similar to device 105 in FIG. 1), one or more medicine containers 210 (only one of which is shown and which can also represent other medical supplies and/or medical devices) which can each include a chip 215 (described herein as an RFID label) that facilitates communications (e.g., bi-directional) between the chip and the device 205 (or other devices) such as within a premises 204. The premises 204 can be various types including a home, a house, an apartment, a room in a building, and so forth. In one embodiment, the device 205 can be part of a smart home assistant which can control various IoT devices and functionality within the premises 204 including security devices (e.g., auto-locks, cameras, alarms, and so forth), lights, thermostats, smart appliances, and so forth.

In one embodiment, the RFID label 215 can include a wireless transceiver which can operate according to various protocols and technology such as Ultra-Wide Band communications. The RFID label 215 can be of various types including passive (e.g., having no battery and obtaining power from a received signal such as from the device 205), semi-passive, and active (e.g., having its own power source such as a battery). Other communication protocols or combinations of protocols can also be utilized by the RFID label 215, such as WiFi and/or Bluetooth®. As an example, the RFID label 215 can include a chip ID for identification of the medicine container 210 (and/or identification of the medication contained therein which may or may not be a separate ID). Other information can also be stored, transmitted and/or received by the RFID label 215 (e.g., to/from the device 205), such as a count of medication that has been dispensed, remaining medication in the medicine container 210, expiration date of the medication, physician information, prescription and dosage information, and so forth.

In one embodiment, healthcare treatment instructions can be pushed to the device 205, such as from equipment or a communication device 220 of a healthcare provider (e.g., a physician, pharmacy, hospital, and so forth). For example, a medication prescription can be pushed from the equipment 220 to the virtual healthcare assistant 205. This instruction can include various information such as medication type, medicine container ID, dosage, and/or schedule. In one embodiment, this push prescription can be coordinated with communications of other equipment such as a pharmacy which can then deliver or otherwise arrange for the medicine container to be provided to the premises 204. In one embodiment, the push prescription from the healthcare provider equipment 220 can be an adjustment to healthcare treatment for the user, such as changing the dosage and/or schedule for medication where the medicine container 210 is already present at the premises 204. In another embodiment, the healthcare treatment instructions can be pushed to the device 205 from the RFID label 215 which may have been provisioned by another device (e.g., provisioned at the pharmacy or physician's office with relevant treatment information including medication ID, container ID, dosage, schedule, and so forth). In this example, one or both of the device 205 and the RFID label 215 can detect the presence of the other through communications (pinging, polling, etc.) or presence can be determined according to manual input entered by someone such as the family of the user if needed or desired.

In one embodiment, the device 205 can include an RFID generator that provisions RFID labels 215 that are to be applied to or are already applied to (or connected with) medicine containers 210. For example, the provisioning can provide information that facilitates the embodiments described herein including locating particular medicine containers 210 having particular medications, wirelessly communicating with a virtual healthcare assistant operating on the customer device 205, determining dispensing of medication from the medicine container, determining a count of dispensed and/or available medication from the medicine container, or combination thereof. The provisioned information can be various types of information including radio IDs, medication IDs, executable code, scheduling information, and so forth.

In one embodiment, healthcare treatment instructions can be pulled by the device 205, such as from the equipment 220 of the healthcare provider or from another source, such as the RFID label 215 which may have been provisioned by another device (e.g., provisioned at the pharmacy or physician's office with relevant treatment information including medication ID, container ID, dosage, schedule, and so forth). For example, a medication prescription can be pulled from the equipment 220 to the virtual healthcare assistant 205, such as based on detecting the presence of a medicine container within the premises 204. This instruction can include various information such as medication type, medicine container ID, dosage, and/or schedule. In one embodiment, this pull prescription can be coordinated and/or verified with communications of other equipment such as a pharmacy which can then deliver or otherwise arrange for the medicine container 210 to be provided to the premises 204. In one embodiment, the pull prescription from the healthcare provider equipment 220 can be an adjustment to healthcare treatment for the user, such as changing the dosage and/or schedule for medication where the medicine container 210 is already present at the premises 204. In one embodiment, one or both of the device 205 and the RFID label 215 can detect the presence of the other through communications (pinging, polling, etc.).

FIG. 2B is a block diagram illustrating an example, non-limiting embodiment of a system 201 functioning within the communication network of FIG. 1 in accordance with various aspects described herein and which can operate in conjunction with the functions of other embodiments described herein including system 200. System 201 illustrates an example of medication dispensing management including navigation and dispensing through use of the customer device 205 (e.g., operating as or otherwise executing the virtual healthcare assistant), and use of the one or more medicine containers 210 (only one of which is shown) which each include the RFID label 215 that facilitates communications between the RFID label and the device 205 such as within the premises 204 which is depicted as having different areas such as a 1^{st} floor kitchen and a 2^{nd} floor bedroom.

In this example, the device 205 can obtain registration information for one or more medicine containers 210, such as via wireless communications with an RFID label 215 of the medicine container when it is detected in the premises 204, scanning of a barcode on the medicine container, user input (e.g., at the device 205), or from other sources.

The device 205 can determine a schedule for dispensing medication and can transmit, to a wearable end user device 230 of the user 225 according to the medication schedule, medication information indicating a reminder to take the medication. In one embodiment, the wearable device 230 can be a smart watch that can include a display and/or audio interface that enables the reminder to be presented to the user 225.

In one embodiment, the device 205 can select a medicine container 210 (from among different containers in the premises) that houses the medication according to the medication schedule and the registration information. A user position of the user 225 in the premises 204 can be determined according to localization messaging (e.g., UWB communications). A container position of the medicine container 210 in the premises 204 can be determined based on communications associated with a wireless transceiver (e.g., RFID label 215) of the medicine container. The particular localization and navigation technique utilized can vary. For example, UWB or other wireless messaging to and/or from one, some, or all of the wearable device 230, the RFID label 215 or the device 205 can be utilized. In one embodiment, the localization messaging can be directly between the RFID label 215 and the wearable device 230, such as where a processor of the wearable device generates navigation instructions based on the localization messaging. In another embodiment, the localization messaging can be between the device 205 and the RFID label 215, as well as between the device 205 and the wearable device 230, such as where the virtual healthcare assistant at device 205 generates navigation instructions based on the localization messaging which are then transmitted to the wearable device 230 for presentation to the user 225.

In the example of FIG. 2B, instructions can be transmitted from the virtual healthcare assistant 205 to the wearable end user device 230 that causes the wearable end user device to present the navigation information that directs the user's movement towards the medicine container 210 (e.g., towards the medicine container 210 located in the 2^{nd} floor bedroom rather than the 1^{st} floor kitchen). As described herein, in one or more embodiments, the navigation instructions can be generated by the wearable device 230 and/or by the virtual healthcare assistant at device 205.

In one embodiment, the device 205 obtains the registration instructions by receiving a wireless message from the RFID label 215 of the medicine container 210. In one embodiment, the device 205 includes an RFID generator which provisions the RFID label 215 with medical data, where the RFID label is already connected with or is configured to be applied to the medicine container 210.

In one embodiment, the device 205 can transmit, to a mobile device of the user 225, other medication information indicating another reminder to take another medication, where the other medication information is transmitted over a wireless network operating at least outside of the premises 204, where the other medication information indicates an identification of another wireless transceiver of another medicine container that houses the other medication to be dispensed. In one embodiment, the mobile device comprises a mobile phone that executes a mobile virtual assistant that is registered with the virtual healthcare assistant operating on the customer device 205 located at the premises 204. Continuing with this example, the device 205 can receive, over the wireless network from the mobile device, dispensing data that was collected by the mobile device from the other wireless transceiver of the other medicine container, where the dispensing data indicates a time of dispensing and an identification of the other medication that was dispensed.

In one embodiment, the device 205 can receive dispensing data from the wireless transceiver of the medicine container 210, where the dispensing data indicates a time of dispensing and an identification of the medication that was dispensed. This information can be shared with other devices, such as equipment 220 of the healthcare provider over network 125. Continuing with this example, the device 205 can add the dispensing data to a medical record of the user 225. In one embodiment, the adding of the dispensing data to the medical record can utilize a blockchain technology. In one embodiment, the device 205 can analyze the medical record utilizing a treatment model to generate a recommendation for when to take the medication; and can transmit instructions to the wearable end user device 230 that causes the wearable end user device to present the recommendation.

In one embodiment, the device 205 can detect another reminder to take the medication and can determine, within another time period of the other reminder, another position of the user 225 in the premises 204 based on other communications associated with the wearable end user device 230 that is worn by the user. Continuing with this example, the device 205 can select a display device from among a group of display devices according to the other position of the user 225 in the premises 204 and can transmit instructions to the display device that causes the display device to present the other reminder.

In one or more embodiments, various dispensing monitoring techniques can be employed to enable the RFID label 2115 to track and report medication that has been dispensed from the medicine container 210. For example, the medicine container 210 can include sensors such as for weighing the contents thereof, for imaging the contents, for determining electrical property changes, and/or for imaging what is being dispensed (where the imaging can be analyzed by the RFID label and/or the device 205 to determine dispensing and/or available medication). In one embodiment, the medicine container 210 can include a physical mechanism for counting pills that have been dispensed, such as a dispensing mechanism that is capable of counting each pill dispensed as a position of the dispensing mechanism is moved to dispense the pill (e.g., a dispensing arm or counter).

FIG. 2C is a block diagram illustrating an example, non-limiting embodiment of a system 202 functioning within the communication network of FIG. 1 in accordance with various aspects described herein and which can operate in conjunction with the functions of other embodiments described herein including systems 200 and/or 201. System 202 illustrates an example of medication dispensing management including navigation and dispensing such as when a user is outside of the premises (e.g., staying with a family member, at a hotel or traveling in a car or train). In one embodiment, this location is depicted as outside of the premises and is further depicted as having different areas such as a 1^{st} floor bedroom and a 2^{nd} floor bathroom. In another embodiment, one or more of the functions described herein could be performed at the premises 204 through use of a mobile device 235 belonging to the user 225 or another individual.

In one embodiment, an end user device 235 of the user 225, can receive medication information indicating a reminder to take the medication. For example, the medication information can be received over the network 125 (or over another network) from the virtual healthcare assistant operating on the customer device 205 located at the premises 204 where the user is normally a resident. In one embodiment, this medication information can indicate an identification of an RFID label 215 of a medicine container 210 that houses the medication to be dispensed. In another embodiment, this medication information can identify a medication to be dispensed from which a medicine container 210 can be identified. A user position of the user 225 (e.g., at a family member's house, a hotel, and so forth) can be determined according to localization messaging (e.g., UWB communications). A container position (e.g., particular room, particular luggage bag, and so forth) of the medicine container 210 can be determined based on communications associated with RFID label 215 of the medicine container. The particular localization and navigation technique utilized can vary and the devices utilized for performing the navigation can also vary.

In one embodiment, the localization messaging can be directly between the RFID label 215 and the user device 235, such as where a smart phone executes a mobile virtual healthcare assistant that generates navigation instructions based on the localization messaging which can then be presented at a display of the user device 235 to facilitate the user 225 locating the medicine container 210. In this example, the user may or may not be wearing the wearable device 230. In another embodiment where the user is wearing the wearable device 230, the localization messaging can be directly between the RFID label 215 and the wearable device 230, such as where a smart phone executes a mobile virtual healthcare assistant that initiates the navigation process being employed by the wearable device based on the localization messaging. In another embodiment, the localization messaging can be between the user device 235 and the RFID label 215, as well as between the user device 235 and the wearable device 230, such as where the virtual healthcare assistant at user device 235 (e.g., a smart phone) generates navigation instructions based on the localization messaging which are then transmitted to the wearable device 230 for presentation to the user 225. As described above, the localization techniques can employ UWB or other wireless messaging to and/or from one, some, or all of the wearable device 230, the RFID label 215, the user device 235 or another device(s).

In another embodiment, a second virtual healthcare assistant can be employed that operates on a customer device 205C where the user is staying (e.g., a family member's house) which is remote from the premises 204. In this example, one or more of the functions of system 201 can be employed (e.g., by device 205C) to facilitate locating the medicine container 210 based on instructions transmitted from the device 205 to the device 205C. This example which employs a second virtual healthcare assistant at the device 205C can be implemented with or without the user device 235 and/or with or without the wearable device 230, such as device 205C causing the user device 235 to generate and/or present the navigation guidance to the user 225 or in another embodiment device 205C causing the wearable device 230 to generate and/or present the navigation guidance to the user 225. As described herein, in one or more embodiments, the localization technique and the navigation instructions can be processed and/or generated by the device 205C, the wearable device 230 and/or the user device 235.

In one embodiment, the user device 235 can obtain registration information for one or more medicine containers 210, such as via wireless communications with an RFID label 215 of the medicine container when it is detected, scanning of a barcode on the medicine container, user input (e.g., at the device 235), or from other sources.

In one embodiment, the user device 235 is a mobile phone (e.g., belonging to the user 225) that executes a mobile virtual healthcare assistant that is registered with a virtual healthcare assistant operating on the customer device 205 located at the premises 204. In this example, the mobile virtual healthcare assistant allows the techniques described herein to be extended outside of the premises 204, such as when the user is staying at a family member's home, a hotel, and so forth. In one or more embodiments, medical information can be collected by various device (e.g., devices 205, 205c, user device 235) and provided to equipment 220 of a healthcare provider via network 125.

In one embodiment, system 202 allows other individuals and their devices to be utilized as part of the rendering of assistance to the user 225. For example, the user device 235 can be a smart phone belonging to someone other than the user 225, such as a family member. The virtual healthcare assistant 205 can determine that the device 235 is in proximity to the user 225 (e.g., location detection applied to the user device 235 and the wearable device 230 or by other techniques) or co-located at the same area that the user is currently present (which may or may not be the premises 204). The reminder notifications and navigation instructions as described herein can then be applied utilizing the device 235 of this other individual so that the other individual can then go and obtain the medicine container 210 for the user 225. In this example, the device 235 may or may not have a mobile virtual healthcare assistant application being executed thereon. For instance, the virtual healthcare assistant 205 can communicate over network 125 with the smart phone 235 of an individual that is present (or will be present) with the user at the time of the scheduled event (e.g., medication dispensing) to provide the notification and to provide information (e.g., ID of the particular RFID label 215) so that the smart phone 235 of this individual can generate localization messaging and present this individual with navigation guidance to move towards the medicine container 210 (e.g., so the individual can bring the medicine container to the user). In this example, different levels of privacy can be maintained (e.g., selected based on user preferences) for the content of the notification message to the individual.

In another embodiment, the device 235 of the individual can communicate with or otherwise be requested by various devices to initiate or facilitate the localization techniques or other healthcare assistance described herein, including being contacted by the second virtual healthcare assistant operating at customer device 205C which has received a request from the virtual healthcare assistant operating at customer device 205 back at the premises 204. In another embodiment, the device 235 of the individual can be utilized to communicate with the wearable device 230 of the user 225 so that the wearable device presents the reminder and presents the navigation guidance directly to the user so that the user can find the medicine container 210 according to any of the localization techniques as described herein.

In one embodiment, system 202 can determine individuals and/or devices that are in proximity to the user at this new location and can select an individual and/or device for facilitating the reminder, localization and/or navigation techniques described herein. For example, a determination can be made (such as by the virtual healthcare assistant 205) that the user is present with a sibling (who has a laptop) and with a child (who has a smart phone). A selection can then be made between the sibling's laptop and the child's smart phone as to which device to communicate with for providing the reminder, localization and navigation. This selection can be made based on various factors including the capabilities of the devices, the relationship of the user to the individual, user preferences, privacy considerations, and so forth. In another embodiment, the device 235 of an individual other than the user can be utilized as described herein when the device 235 and the user 225 are present at the premises 204 (i.e., the user has not left the premises) so that the individual can assist the user.

FIG. 2D is a block diagram illustrating an example, non-limiting embodiment of a system 203 functioning within the communication network of FIG. 1 in accordance with various aspects described herein and which can operate in conjunction with the functions of other embodiments described herein including one or more of systems 200-202. System 203 illustrates an example of healthcare appointment management including navigation and conferencing through use of the customer device 205 (e.g., operating as or otherwise executing the virtual healthcare assistant), where the device 205 is within the premises 204 which is depicted as having different areas such as a 1^{st} floor kitchen and a 2^{nd} floor bedroom.

In one or more embodiments, a scheduled appointment can be identified by the device 205 that is to occur at a future time for the user 225. The device 205 can determine, within a time period of the scheduled appointment, a position of the user 225 in the premises 204 based on communications (e.g., UWB messaging) associated with the wearable end user device 230 that is worn by the user. The device 205 can select a display device 240A from among a group of display devices 240A, B (e.g., any number of display devices) according to the position of the user in the premises 204 (e.g., a closest display device, a display device that does not require travelling up to a different floor, and so forth). In one embodiment, the positions of the display devices 240 can be known to the device 205 and/or can be detected by the device 205, such as via localization techniques. In one embodiment, the selection of the display device can be based on other factors including capabilities of the display device, current usage of the display device, user preferences, privacy considerations, and so forth. In one embodiment, the device 205 can facilitate establishing a video conference with a communication device 220 of the healthcare provider so that the video conference is presented at the display device 240A. The video conference can be established using various protocols and techniques including the display device 240A connecting directly with the communication device 220 via the network 125 or the device 205 connecting with the communication device 220 via the network 125 and presenting/capturing video and audio at the display device 240A. In one embodiment, the display device 240a includes a camera and audio device for capturing video and audio of the user 225.

In one embodiment, dispensing data can be received (e.g., by the device 205 over a wireless network operating in the premises 204) from a wireless transmitter of a medicine container, where the dispensing data indicates a time of dispensing and/or an identification of medication that was dispensed. In this example, the dispensing data or a portion thereof can be transmitted over the network 125 to the communication device 220 associated with a healthcare provider. In one embodiment, the device 205 can transmit instructions to the wearable end user device 230 that causes the wearable end user device to present navigation information that directs movement towards the display device 240A that was selected.

In one embodiment, the device 205 can transmit, to an end user device (e.g., the wearable device 230, a smart phone, and so forth) of the user 225, medication information indicating a reminder to take a particular where the medication information is transmitted over the network 125. In this example, the medication information can indicate an identification of a wireless transceiver of a particular medicine container that houses the medication to be dispensed. In one embodiment, the end user device receiving the medication information is a mobile phone that executes a mobile virtual healthcare assistant that is registered with a virtual healthcare assistant operating on the device 205 located at the premises 204. In one embodiment, the device 205 can receive dispensing data that was collected by the end user device from the wireless transceiver of the medicine container, where the dispensing data indicates a time of dispensing and an identification of the medication that was dispensed.

In one embodiment, the management of the virtual doctor's appointment can occur at a location outside of the premises. For example, the second virtual healthcare assistant 205C at a different location (where the user is present) can communicate with the virtual healthcare assistant 205 at the premises 204 and can coordinate the functions described above with respect to selecting a display device, facilitating establishing a video conference, providing navigation guidance to the user 225 towards the selected display device, and so forth. In another embodiment, the device 235 (e.g., a smart phone) of the user or of another individual at the different location (where the user is present) can communicate with the virtual healthcare assistant 205 at the premises 204 and can coordinate the functions described above with respect to selecting a display device, facilitating establishing a video conference, providing navigation guidance to the user 225 towards the selected display device, and so forth. In one embodiment, the management of the virtual doctor's appointment can occur while the user 225 is travelling such as the virtual healthcare assistant operating at device 205 at the premises 204 determining that the user is not present at the premises for a scheduled doctor's appointment and obtaining a location of the user (e.g., travelling in a car). Continuing with this example, the virtual healthcare assistant 205 can select a display device for conducting the video conference with the equipment 220 of the healthcare provider. In this example, the selection can be a mobile device (e.g., mobile device 235 of system 202 of FIG. 2C which may belong to the user or to another individual) in the car with the user 225. The video conference can then be established as described herein.

FIG. 2E depicts an illustrative embodiment of a method 250 in accordance with various aspects described herein. At 252, one or more medicine containers can be registered with a virtual healthcare assistant (e.g., the virtual healthcare assistant operating on device 205, 205c or mobile device 235). The registration can be performed utilizing various techniques including wireless communications, scanning barcodes, user input, imaging, and so forth. At 254, a reminder is transmitted. For example, a processing system of an end user device of a user can receive medication information indicating a reminder to take a medication. The end user device can be a wearable device such as a smart watch or can be other types of devices such as a mobile device or a smart phone. The medication information can be received over a wireless network from the virtual healthcare assistant operating on a customer device located at a premises where a user is present or is a resident. In one embodiment, the medication information can indicate an identification of a wireless transceiver of a medicine container that houses the medication to be dispensed. At 256, various location techniques are performed. For example, the end user device can determine a user position of the user (which may or may not be remote from the premises) such as based on communications (e.g., UWB messaging). Continuing with this example, the end user device can determine a container position of the medicine container (which may or may not be remote from the premises) such as based on communications (e.g., UWB messaging) with the wireless transceiver of the medicine container. At 258, the end user device can present navigation information that facilitates movement by the user towards the medicine container, such as at a display of the end user device, audio instructions, and so forth.

While for purposes of simplicity of explanation, the respective processes are shown and described as a series of blocks in FIG. 2E, it is to be understood and appreciated that the claimed subject matter is not limited by the order of the blocks, as some blocks may occur in different orders and/or concurrently with other blocks from what is depicted and described herein. Moreover, not all illustrated blocks may be required to implement the methods described herein.

In one or more embodiments, the virtual healthcare assistant operating at device 205 can collect data which can be utilized in improving the healthcare treatment of the user. As described herein, medication dispensing can be tracked and placed into medical records, as well as managing supplies of the medication, such as directly with the pharmacy and/or healthcare provider. In one embodiment, other information can be collected with respect to the user including biometrics, user feedback, scheduled activities, and so forth, which can then be analyzed, such as via a trained model, to make recommendations. For instance, the trained model can determine (based on collected data) that a time for taking medication can be changed because a user feels better when taking it in the afternoon rather than in the morning.

In one or more embodiments, the information being communicated can be confidential such that authentication between various devices (e.g., customer device 205, wearable device 230, end user device 235, healthcare provider equipment 220, and so forth) is required and performed.

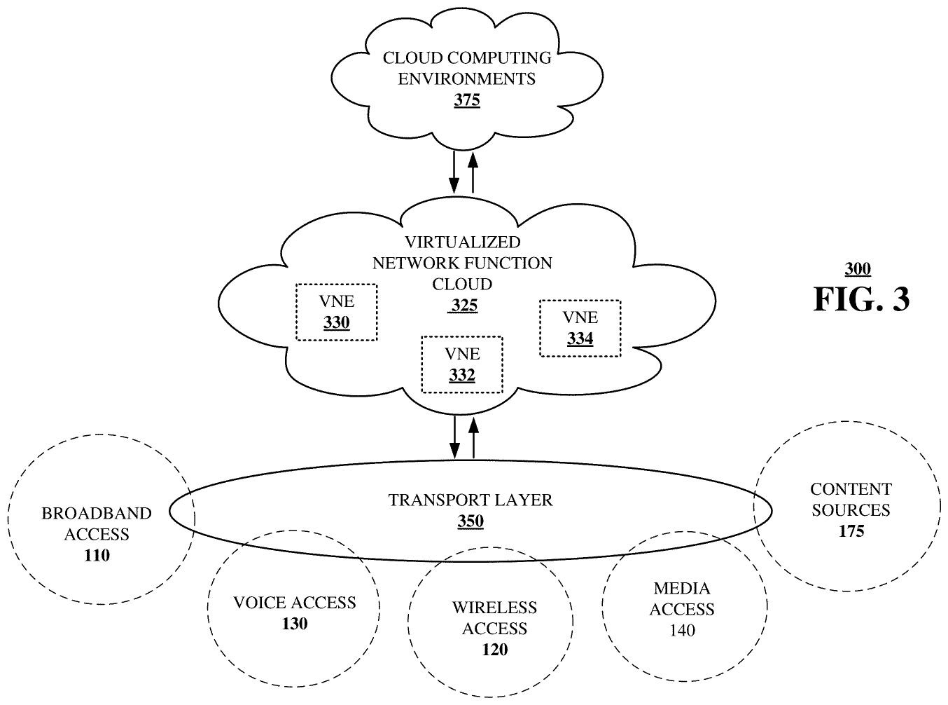

Referring now to FIG. 3, a block diagram 300 is shown illustrating an example, non-limiting embodiment of a virtualized communication network in accordance with various aspects described herein. In particular a virtualized communication network is presented that can be used to implement some or all of the subsystems and functions of system 100, the subsystems and functions of system 200, and method 250 presented in FIGS. 1, 2A-2E, and 3. For example, virtualized communication network 300 can facilitate in whole or in part providing a virtual healthcare assistant to facilitate providing healthcare to a user such as by determining a scheduled appointment at a future time for a user that is a resident of a premises in which the device is located; receiving, over a wireless network operating in the premises, dispensing data from a wireless transmitter of a medicine container, the dispensing data indicating a time of dispensing and an identification of medication that was dispensed; transmitting the dispensing data to a communication device associated with a healthcare provider; determining, within a time period of the scheduled appointment, a position of the user in the premises based on communications associated with a wearable end user device that is worn by the user; selecting a display device from among a group of display devices according to the position of the user in the premises; and facilitating establishing a video conference with the communication device of the healthcare provider that is presented at the display device of the user.

In particular, a cloud networking architecture is shown that leverages cloud technologies and supports rapid innovation and scalability via a transport layer 350, a virtualized network function cloud 325 and/or one or more cloud computing environments 375. In various embodiments, this cloud networking architecture is an open architecture that leverages application programming interfaces (APIs); reduces complexity from services and operations; supports more nimble business models; and rapidly and seamlessly scales to meet evolving customer requirements including traffic growth, diversity of traffic types, and diversity of performance and reliability expectations.

In contrast to traditional network elements—which are typically integrated to perform a single function, the virtualized communication network employs virtual network elements (VNEs) 330, 332, 334, etc. that perform some or all of the functions of network elements 150, 152, 154, 156, etc. For example, the network architecture can provide a substrate of networking capability, often called Network Function Virtualization Infrastructure (NFVI) or simply infrastructure that is capable of being directed with software and Software Defined Networking (SDN) protocols to perform a broad variety of network functions and services. This infrastructure can include several types of substrates. The most typical type of substrate being servers that support Network Function Virtualization (NFV), followed by packet forwarding capabilities based on generic computing resources, with specialized network technologies brought to bear when general purpose processors or general purpose integrated circuit devices offered by merchants (referred to herein as merchant silicon) are not appropriate. In this case, communication services can be implemented as cloud-centric workloads.

As an example, a traditional network element 150 (shown in FIG. 1), such as an edge router can be implemented via a VNE 330 composed of NFV software modules, merchant silicon, and associated controllers. The software can be written so that increasing workload consumes incremental resources from a common resource pool, and moreover so that it's elastic: so the resources are only consumed when needed. In a similar fashion, other network elements such as other routers, switches, edge caches, and middle-boxes are instantiated from the common resource pool. Such sharing of infrastructure across a broad set of uses makes planning and growing infrastructure easier to manage.

In an embodiment, the transport layer 350 includes fiber, cable, wired and/or wireless transport elements, network elements and interfaces to provide broadband access 110, wireless access 120, voice access 130, media access 140 and/or access to content sources 175 for distribution of content to any or all of the access technologies. In particular, in some cases a network element needs to be positioned at a specific place, and this allows for less sharing of common infrastructure. Other times, the network elements have specific physical layer adapters that cannot be abstracted or virtualized, and might require special DSP code and analog front-ends (AFEs) that do not lend themselves to implementation as VNEs 330, 332 or 334. These network elements can be included in transport layer 350.

The virtualized network function cloud 325 interfaces with the transport layer 350 to provide the VNEs 330, 332, 334, etc. to provide specific NFVs. In particular, the virtualized network function cloud 325 leverages cloud operations, applications, and architectures to support networking workloads. The virtualized network elements 330, 332 and 334 can employ network function software that provides either a one-for-one mapping of traditional network element function or alternately some combination of network functions designed for cloud computing. For example, VNEs 330, 332 and 334 can include route reflectors, domain name system (DNS) servers, and dynamic host configuration protocol (DHCP) servers, system architecture evolution (SAE) and/or mobility management entity (MME) gateways, broadband network gateways, IP edge routers for IP-VPN, Ethernet and other services, load balancers, distributers and other network elements. Because these elements don't typically need to forward large amounts of traffic, their workload can be distributed across a number of servers—each of which adds a portion of the capability, and overall which creates an elastic function with higher availability than its former monolithic version. These virtual network elements 330, 332, 334, etc. can be instantiated and managed using an orchestration approach similar to those used in cloud compute services.

The cloud computing environments 375 can interface with the virtualized network function cloud 325 via APIs that expose functional capabilities of the VNEs 330, 332, 334, etc. to provide the flexible and expanded capabilities to the virtualized network function cloud 325. In particular, network workloads may have applications distributed across the virtualized network function cloud 325 and cloud computing environment 375 and in the commercial cloud, or might simply orchestrate workloads supported entirely in NFV infrastructure from these third party locations.

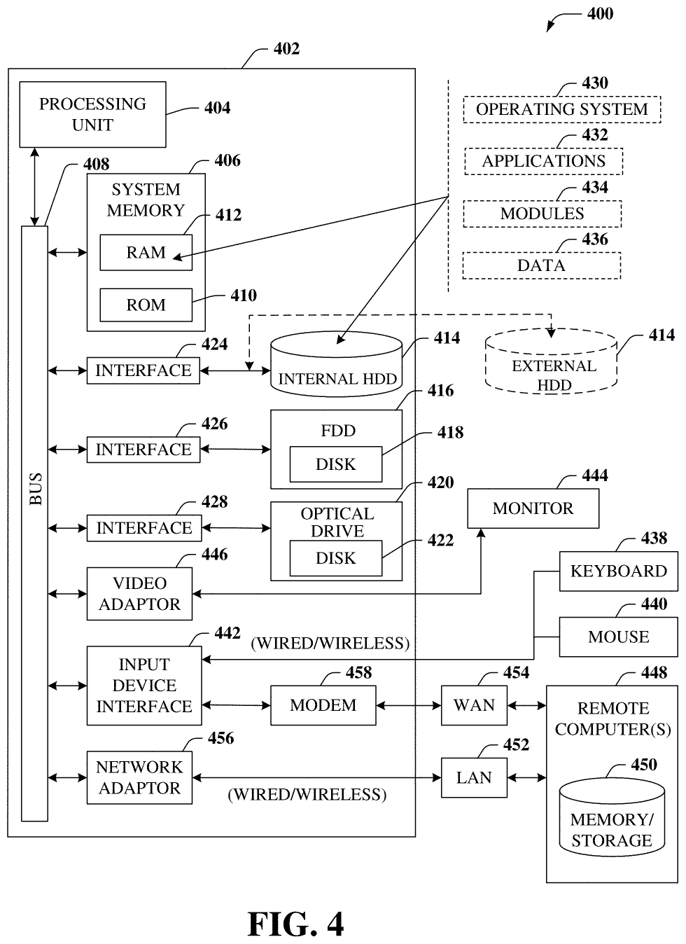

Turning now to FIG. 4, there is illustrated a block diagram of a computing environment in accordance with various aspects described herein. In order to provide additional context for various embodiments of the embodiments described herein, FIG. 4 and the following discussion are intended to provide a brief, general description of a suitable computing environment 400 in which the various embodiments of the subject disclosure can be implemented. In particular, computing environment 400 can be used in the implementation of network elements 150, 152, 154, 156, access terminal 112, base station or access point 122, switching device 132, media terminal 142, and/or VNEs 330, 332, 334, etc. Each of these devices can be implemented via computer-executable instructions that can run on one or more computers, and/or in combination with other program modules and/or as a combination of hardware and software. For example, computing environment 400 can facilitate in whole or in part providing a virtual healthcare assistant to facilitate providing healthcare to a user such as by obtaining registration information for a group of medicine containers that house different medications; transmitting, to a wearable end user device of the user according to a medication schedule, medication information indicating a reminder to take a medication; selecting a medicine container that houses the medication from among the group of medicine containers according to the medication schedule and the registration information; and transmitting instructions to the wearable end user device that causes the wearable end user device to present navigation information that directs movement of the user towards the medicine container, wherein the navigation instructions are based on localization messaging associated with the wearable end user device and an RFID label of the medicine container.

Generally, program modules comprise routines, programs, components, data structures, etc., that perform particular tasks or implement particular abstract data types. Moreover, those skilled in the art will appreciate that the methods can be practiced with other computer system configurations, comprising single-processor or multiprocessor computer systems, minicomputers, mainframe computers, as well as personal computers, hand-held computing devices, microprocessor-based or programmable consumer electronics, and the like, each of which can be operatively coupled to one or more associated devices.

As used herein, a processing circuit includes one or more processors as well as other application specific circuits such as an application specific integrated circuit, digital logic circuit, state machine, programmable gate array or other circuit that processes input signals or data and that produces output signals or data in response thereto. It should be noted that while any functions and features described herein in association with the operation of a processor could likewise be performed by a processing circuit.

The illustrated embodiments of the embodiments herein can be also practiced in distributed computing environments where certain tasks are performed by remote processing devices that are linked through a communications network. In a distributed computing environment, program modules can be located in both local and remote memory storage devices.

Computing devices typically comprise a variety of media, which can comprise computer-readable storage media and/or communications media, which two terms are used herein differently from one another as follows. Computer-readable storage media can be any available storage media that can be accessed by the computer and comprises both volatile and nonvolatile media, removable and non-removable media. By way of example, and not limitation, computer-readable storage media can be implemented in connection with any method or technology for storage of information such as computer-readable instructions, program modules, structured data or unstructured data.

Computer-readable storage media can comprise, but are not limited to, random access memory (RAM), read only memory (ROM), electrically erasable programmable read only memory (EEPROM), flash memory or other memory technology, compact disk read only memory (CD-ROM), digital versatile disk (DVD) or other optical disk storage, magnetic cassettes, magnetic tape, magnetic disk storage or other magnetic storage devices or other tangible and/or non-transitory media which can be used to store desired information. In this regard, the terms “tangible” or “non-transitory” herein as applied to storage, memory or computer-readable media, are to be understood to exclude only propagating transitory signals per se as modifiers and do not relinquish rights to all standard storage, memory or computer-readable media that are not only propagating transitory signals per se.

Computer-readable storage media can be accessed by one or more local or remote computing devices, e.g., via access requests, queries or other data retrieval protocols, for a variety of operations with respect to the information stored by the medium.

Communications media typically embody computer-readable instructions, data structures, program modules or other structured or unstructured data in a data signal such as a modulated data signal, e.g., a carrier wave or other transport mechanism, and comprises any information delivery or transport media. The term “modulated data signal” or signals refers to a signal that has one or more of its characteristics set or changed in such a manner as to encode information in one or more signals. By way of example, and not limitation, communication media comprise wired media, such as a wired network or direct-wired connection, and wireless media such as acoustic, RF, infrared and other wireless media.

With reference again to FIG. 4, the example environment can comprise a computer 402, the computer 402 comprising a processing unit 404, a system memory 406 and a system bus 408. The system bus 408 couples system components including, but not limited to, the system memory 406 to the processing unit 404. The processing unit 404 can be any of various commercially available processors. Dual microprocessors and other multiprocessor architectures can also be employed as the processing unit 404.

The system bus 408 can be any of several types of bus structure that can further interconnect to a memory bus (with or without a memory controller), a peripheral bus, and a local bus using any of a variety of commercially available bus architectures. The system memory 406 comprises ROM 410 and RAM 412. A basic input/output system (BIOS) can be stored in a non-volatile memory such as ROM, erasable programmable read only memory (EPROM), EEPROM, which BIOS contains the basic routines that help to transfer information between elements within the computer 402, such as during startup. The RAM 412 can also comprise a high-speed RAM such as static RAM for caching data.

The computer 402 further comprises an internal hard disk drive (HDD) 414 (e.g., EIDE, SATA), which internal HDD 414 can also be configured for external use in a suitable chassis (not shown), a magnetic floppy disk drive (FDD) 416, (e.g., to read from or write to a removable diskette 418) and an optical disk drive 420, (e.g., reading a CD-ROM disk 422 or, to read from or write to other high capacity optical media such as the DVD). The HDD 414, magnetic FDD 416 and optical disk drive 420 can be connected to the system bus 408 by a hard disk drive interface 424, a magnetic disk drive interface 426 and an optical drive interface 428, respectively. The hard disk drive interface 424 for external drive implementations comprises at least one or both of Universal Serial Bus (USB) and Institute of Electrical and Electronics Engineers (IEEE) 1394 interface technologies. Other external drive connection technologies are within contemplation of the embodiments described herein.

The drives and their associated computer-readable storage media provide nonvolatile storage of data, data structures, computer-executable instructions, and so forth. For the computer 402, the drives and storage media accommodate the storage of any data in a suitable digital format. Although the description of computer-readable storage media above refers to a hard disk drive (HDD), a removable magnetic diskette, and a removable optical media such as a CD or DVD, it should be appreciated by those skilled in the art that other types of storage media which are readable by a computer, such as zip drives, magnetic cassettes, flash memory cards, cartridges, and the like, can also be used in the example operating environment, and further, that any such storage media can contain computer-executable instructions for performing the methods described herein.

A number of program modules can be stored in the drives and RAM 412, comprising an operating system 430, one or more application programs 432, other program modules 434 and program data 436. All or portions of the operating system, applications, modules, and/or data can also be cached in the RAM 412. The systems and methods described herein can be implemented utilizing various commercially available operating systems or combinations of operating systems.

A user can enter commands and information into the computer 402 through one or more wired/wireless input devices, e.g., a keyboard 438 and a pointing device, such as a mouse 440. Other input devices (not shown) can comprise a microphone, an infrared (IR) remote control, a joystick, a game pad, a stylus pen, touch screen or the like. These and other input devices are often connected to the processing unit 404 through an input device interface 442 that can be coupled to the system bus 408, but can be connected by other interfaces, such as a parallel port, an IEEE 1394 serial port, a game port, a universal serial bus (USB) port, an IR interface, etc.

A monitor 444 or other type of display device can be also connected to the system bus 408 via an interface, such as a video adapter 446. It will also be appreciated that in alternative embodiments, a monitor 444 can also be any display device (e.g., another computer having a display, a smart phone, a tablet computer, etc.) for receiving display information associated with computer 402 via any communication means, including via the Internet and cloud-based networks. In addition to the monitor 444, a computer typically comprises other peripheral output devices (not shown), such as speakers, printers, etc.

The computer 402 can operate in a networked environment using logical connections via wired and/or wireless communications to one or more remote computers, such as a remote computer(s) 448. The remote computer(s) 448 can be a workstation, a server computer, a router, a personal computer, portable computer, microprocessor-based entertainment appliance, a peer device or other common network node, and typically comprises many or all of the elements described relative to the computer 402, although, for purposes of brevity, only a remote memory/storage device 450 is illustrated. The logical connections depicted comprise wired/wireless connectivity to a local area network (LAN) 452 and/or larger networks, e.g., a wide area network (WAN) 454. Such LAN and WAN networking environments are commonplace in offices and companies, and facilitate enterprise-wide computer networks, such as intranets, all of which can connect to a global communications network, e.g., the Internet.

When used in a LAN networking environment, the computer 402 can be connected to the LAN 452 through a wired and/or wireless communication network interface or adapter 456. The adapter 456 can facilitate wired or wireless communication to the LAN 452, which can also comprise a wireless AP disposed thereon for communicating with the adapter 456.

When used in a WAN networking environment, the computer 402 can comprise a modem 458 or can be connected to a communications server on the WAN 454 or has other means for establishing communications over the WAN 454, such as by way of the Internet. The modem 458, which can be internal or external and a wired or wireless device, can be connected to the system bus 408 via the input device interface 442. In a networked environment, program modules depicted relative to the computer 402 or portions thereof, can be stored in the remote memory/storage device 450. It will be appreciated that the network connections shown are example and other means of establishing a communications link between the computers can be used.

The computer 402 can be operable to communicate with any wireless devices or entities operatively disposed in wireless communication, e.g., a printer, scanner, desktop and/or portable computer, portable data assistant, communications satellite, any piece of equipment or location associated with a wirelessly detectable tag (e.g., a kiosk, news stand, restroom), and telephone. This can comprise Wireless Fidelity (Wi-Fi) and BLUETOOTH® wireless technologies. Thus, the communication can be a predefined structure as with a conventional network or simply an ad hoc communication between at least two devices.

Wi-Fi can allow connection to the Internet from a couch at home, a bed in a hotel room or a conference room at work, without wires. Wi-Fi is a wireless technology similar to that used in a cell phone that enables such devices, e.g., computers, to send and receive data indoors and out; anywhere within the range of a base station. Wi-Fi networks use radio technologies called IEEE 802.11 (a, b, g, n, ac, ag, etc.) to provide secure, reliable, fast wireless connectivity. A Wi-Fi network can be used to connect computers to each other, to the Internet, and to wired networks (which can use IEEE 802.3 or Ethernet). Wi-Fi networks operate in the unlicensed 2.4 and 5 GHz radio bands for example or with products that contain both bands (dual band), so the networks can provide real-world performance similar to the basic 10BaseT wired Ethernet networks used in many offices.

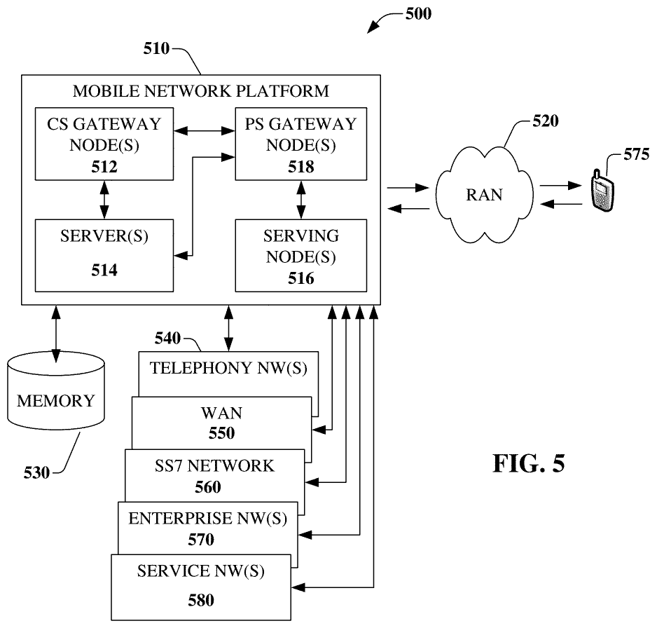

Turning now to FIG. 5, an embodiment 500 of a mobile network platform 510 is shown that is an example of network elements 150, 152, 154, 156, and/or VNEs 330, 332, 334, etc. For example, platform 510 can facilitate in whole or in part providing a virtual healthcare assistant to facilitate providing healthcare to a user such as by receiving, by a processing system including a processor of an end user device of a user, medication information indicating a reminder to take a medication, the medication information being received over a wireless network from a virtual healthcare assistant operating on a customer device located at a premises where a user is a resident, the medication information indicating an identification of a wireless transceiver of a medicine container that houses the medication to be dispensed; determining, by the processing system, a user position of the user that is remote from the premises; determining, by the processing system, a container position of the medicine container that is remote from the premises based on communications with the wireless transceiver of the medicine container; and presenting, by the processing system at a display of the end user device, navigation information that facilitates movement by the user towards the medicine container.

In one or more embodiments, the mobile network platform 510 can generate and receive signals transmitted and received by base stations or access points such as base station or access point 122. Generally, mobile network platform 510 can comprise components, e.g., nodes, gateways, interfaces, servers, or disparate platforms, that facilitate both packet-switched (PS) (e.g., internet protocol (IP), frame relay, asynchronous transfer mode (ATM)) and circuit-switched (CS) traffic (e.g., voice and data), as well as control generation for networked wireless telecommunication. As a non-limiting example, mobile network platform 510 can be included in telecommunications carrier networks, and can be considered carrier-side components as discussed elsewhere herein. Mobile network platform 510 comprises CS gateway node(s) 512 which can interface CS traffic received from legacy networks like telephony network(s) 540 (e.g., public switched telephone network (PSTN), or public land mobile network (PLMN)) or a signaling system #7 (SS7) network 560. CS gateway node(s) 512 can authorize and authenticate traffic (e.g., voice) arising from such networks. Additionally, CS gateway node(s) 512 can access mobility, or roaming, data generated through SS7 network 560; for instance, mobility data stored in a visited location register (VLR), which can reside in memory 530. Moreover, CS gateway node(s) 512 interfaces CS-based traffic and signaling and PS gateway node(s) 518. As an example, in a 3GPP UMTS network, CS gateway node(s) 512 can be realized at least in part in gateway GPRS support node(s) (GGSN). It should be appreciated that functionality and specific operation of CS gateway node(s) 512, PS gateway node(s) 518, and serving node(s) 516, is provided and dictated by radio technology(ies) utilized by mobile network platform 510 for telecommunication over a radio access network 520 with other devices, such as a radiotelephone 575.

In addition to receiving and processing CS-switched traffic and signaling, PS gateway node(s) 518 can authorize and authenticate PS-based data sessions with served mobile devices. Data sessions can comprise traffic, or content(s), exchanged with networks external to the mobile network platform 510, like wide area network(s) (WANs) 550, enterprise network(s) 570, and service network(s) 580, which can be embodied in local area network(s) (LANs), can also be interfaced with mobile network platform 510 through PS gateway node(s) 518. It is to be noted that WANs 550 and enterprise network(s) 570 can embody, at least in part, a service network(s) like IP multimedia subsystem (IMS). Based on radio technology layer(s) available in technology resource(s) or radio access network 520, PS gateway node(s) 518 can generate packet data protocol contexts when a data session is established; other data structures that facilitate routing of packetized data also can be generated. To that end, in an aspect, PS gateway node(s) 518 can comprise a tunnel interface (e.g., tunnel termination gateway (TTG) in 3GPP UMTS network(s) (not shown)) which can facilitate packetized communication with disparate wireless network(s), such as Wi-Fi networks.

In embodiment 500, mobile network platform 510 also comprises serving node(s) 516 that, based upon available radio technology layer(s) within technology resource(s) in the radio access network 520, convey the various packetized flows of data streams received through PS gateway node(s) 518. It is to be noted that for technology resource(s) that rely primarily on CS communication, server node(s) can deliver traffic without reliance on PS gateway node(s) 518; for example, server node(s) can embody at least in part a mobile switching center. As an example, in a 3GPP UMTS network, serving node(s) 516 can be embodied in serving GPRS support node(s) (SGSN).

For radio technologies that exploit packetized communication, server(s) 514 in mobile network platform 510 can execute numerous applications that can generate multiple disparate packetized data streams or flows, and manage (e.g., schedule, queue, format . . . ) such flows. Such application(s) can comprise add-on features to standard services (for example, provisioning, billing, customer support . . . ) provided by mobile network platform 510. Data streams (e.g., content(s) that are part of a voice call or data session) can be conveyed to PS gateway node(s) 518 for authorization/authentication and initiation of a data session, and to serving node(s) 516 for communication thereafter. In addition to application server, server(s) 514 can comprise utility server(s), a utility server can comprise a provisioning server, an operations and maintenance server, a security server that can implement at least in part a certificate authority and firewalls as well as other security mechanisms, and the like. In an aspect, security server(s) secure communication served through mobile network platform 510 to ensure network's operation and data integrity in addition to authorization and authentication procedures that CS gateway node(s) 512 and PS gateway node(s) 518 can enact. Moreover, provisioning server(s) can provision services from external network(s) like networks operated by a disparate service provider; for instance, WAN 550 or Global Positioning System (GPS) network(s) (not shown). Provisioning server(s) can also provision coverage through networks associated to mobile network platform 510 (e.g., deployed and operated by the same service provider), such as the distributed antennas networks shown in FIG. 1(s) that enhance wireless service coverage by providing more network coverage.

It is to be noted that server(s) 514 can comprise one or more processors configured to confer at least in part the functionality of mobile network platform 510. To that end, the one or more processor can execute code instructions stored in memory 530, for example. It is should be appreciated that server(s) 514 can comprise a content manager, which operates in substantially the same manner as described hereinbefore.

In example embodiment 500, memory 530 can store information related to operation of mobile network platform 510. Other operational information can comprise provisioning information of mobile devices served through mobile network platform 510, subscriber databases; application intelligence, pricing schemes, e.g., promotional rates, flat-rate programs, couponing campaigns; technical specification(s) consistent with telecommunication protocols for operation of disparate radio, or wireless, technology layers; and so forth. Memory 530 can also store information from at least one of telephony network(s) 540, WAN 550, SS7 network 560, or enterprise network(s) 570. In an aspect, memory 530 can be, for example, accessed as part of a data store component or as a remotely connected memory store.

In order to provide a context for the various aspects of the disclosed subject matter, FIG. 5, and the following discussion, are intended to provide a brief, general description of a suitable environment in which the various aspects of the disclosed subject matter can be implemented. While the subject matter has been described above in the general context of computer-executable instructions of a computer program that runs on a computer and/or computers, those skilled in the art will recognize that the disclosed subject matter also can be implemented in combination with other program modules. Generally, program modules comprise routines, programs, components, data structures, etc. that perform particular tasks and/or implement particular abstract data types.

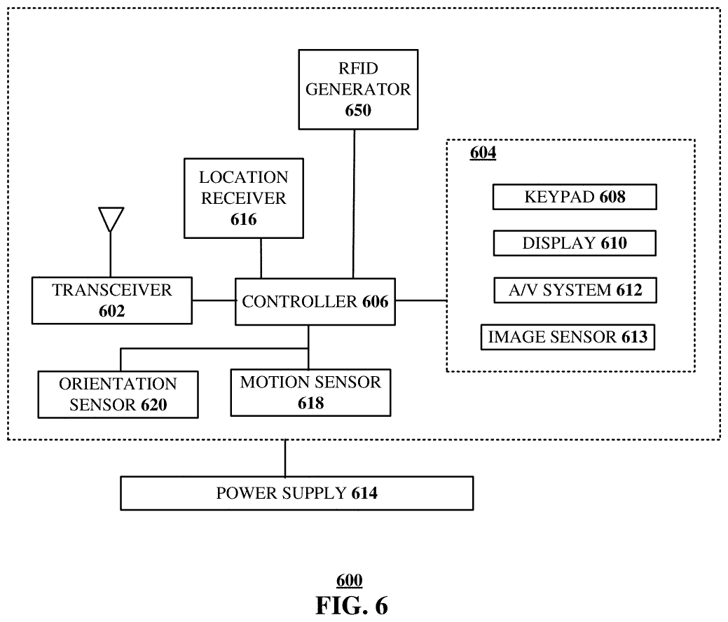

Turning now to FIG. 6, an illustrative embodiment of a communication device 600 is shown. The communication device 600 can serve as an illustrative embodiment of devices such as data terminals 114, mobile devices 124, vehicle 126, display devices 144 or other client devices for communication via either communications network 125. For example, computing device 600 can facilitate in whole or in part providing a virtual healthcare assistant to facilitate providing healthcare to a user such as by determining a scheduled appointment at a future time for a user that is a resident of a premises in which the device is located; receiving, over a wireless network operating in the premises, dispensing data from a wireless transmitter of a medicine container, the dispensing data indicating a time of dispensing and an identification of medication that was dispensed; transmitting the dispensing data to a communication device associated with a healthcare provider; determining, within a time period of the scheduled appointment, a position of the user in the premises based on communications associated with a wearable end user device that is worn by the user; selecting a display device from among a group of display devices according to the position of the user in the premises; and facilitating establishing a video conference with the communication device of the healthcare provider that is presented at the display device of the user.

The communication device 600 can comprise a wireline and/or wireless transceiver 602 (herein transceiver 602), a user interface (UI) 604, a power supply 614, a location receiver 616, a motion sensor 618, an orientation sensor 620, and a controller 606 for managing operations thereof. The transceiver 602 can support short-range or long-range wireless access technologies such as Bluetooth®, ZigBee®, WiFi, DECT, or cellular communication technologies, just to mention a few (Bluetooth® and ZigBee® are trademarks registered by the Bluetooth® Special Interest Group and the ZigBee® Alliance, respectively). Cellular technologies can include, for example, CDMA-1X, UMTS/HSDPA, GSM/GPRS, TDMA/EDGE, EV/DO, WiMAX, SDR, LTE, as well as other next generation wireless communication technologies as they arise. The transceiver 602 can also be adapted to support circuit-switched wireline access technologies (such as PSTN), packet-switched wireline access technologies (such as TCP/IP, VoIP, etc.), and combinations thereof.

The UI 604 can include a depressible or touch-sensitive keypad 608 with a navigation mechanism such as a roller ball, a joystick, a mouse, or a navigation disk for manipulating operations of the communication device 600. The keypad 608 can be an integral part of a housing assembly of the communication device 600 or an independent device operably coupled thereto by a tethered wireline interface (such as a USB cable) or a wireless interface supporting for example Bluetooth®. The keypad 608 can represent a numeric keypad commonly used by phones, and/or a QWERTY keypad with alphanumeric keys. The UI 604 can further include a display 610 such as monochrome or color LCD (Liquid Crystal Display), OLED (Organic Light Emitting Diode) or other suitable display technology for conveying images to an end user of the communication device 600. In an embodiment where the display 610 is touch-sensitive, a portion or all of the keypad 608 can be presented by way of the display 610 with navigation features.

The display 610 can use touch screen technology to also serve as a user interface for detecting user input. As a touch screen display, the communication device 600 can be adapted to present a user interface having graphical user interface (GUI) elements that can be selected by a user with a touch of a finger. The display 610 can be equipped with capacitive, resistive or other forms of sensing technology to detect how much surface area of a user's finger has been placed on a portion of the touch screen display. This sensing information can be used to control the manipulation of the GUI elements or other functions of the user interface. The display 610 can be an integral part of the housing assembly of the communication device 600 or an independent device communicatively coupled thereto by a tethered wireline interface (such as a cable) or a wireless interface.

The UI 604 can also include an audio system 612 that utilizes audio technology for conveying low volume audio (such as audio heard in proximity of a human ear) and high volume audio (such as speakerphone for hands free operation). The audio system 612 can further include a microphone for receiving audible signals of an end user. The audio system 612 can also be used for voice recognition applications. The UI 604 can further include an image sensor 613 such as a charged coupled device (CCD) camera for capturing still or moving images.

The power supply 614 can utilize common power management technologies such as replaceable and rechargeable batteries, supply regulation technologies, and/or charging system technologies for supplying energy to the components of the communication device 600 to facilitate long-range or short-range portable communications. Alternatively, or in combination, the charging system can utilize external power sources such as DC power supplied over a physical interface such as a USB port or other suitable tethering technologies.

The location receiver 616 can utilize location technology such as a global positioning system (GPS) receiver capable of assisted GPS for identifying a location of the communication device 600 based on signals generated by a constellation of GPS satellites, which can be used for facilitating location services such as navigation. The motion sensor 618 can utilize motion sensing technology such as an accelerometer, a gyroscope, or other suitable motion sensing technology to detect motion of the communication device 600 in three-dimensional space. The orientation sensor 620 can utilize orientation sensing technology such as a magnetometer to detect the orientation of the communication device 600 (north, south, west, and east, as well as combined orientations in degrees, minutes, or other suitable orientation metrics).

The communication device 600 can use the transceiver 602 to also determine a proximity to a cellular, WiFi, Bluetooth®, or other wireless access points by sensing techniques such as utilizing a received signal strength indicator (RSSI) and/or signal time of arrival (TOA) or time of flight (TOF) measurements. The controller 606 can utilize computing technologies such as a microprocessor, a digital signal processor (DSP), programmable gate arrays, application specific integrated circuits, and/or a video processor with associated storage memory such as Flash, ROM, RAM, SRAM, DRAM or other storage technologies for executing computer instructions, controlling, and processing data supplied by the aforementioned components of the communication device 600.

The communication device 600 can include an RFID generator 650 that provisions RFID labels that are to be applied to or are already applied to (or connected with) medicine containers or other medical supplies/devices. As an example, the RFID generator 650 can be a device that can individually provision RFID labels housed (e.g., loaded into) by communication device 600 where the RFID labels are connectable (e.g., a self-adhesive side) to medicine containers and where the provisioning provides information that facilitates the embodiments described herein including locating particular medicine containers having particular medications, wirelessly communicating with a virtual healthcare assistant operating on a customer device located at a premises where a user is a resident or operating on a smart phone, determining dispensing of and availability of medication from the medicine container, determining a count of dispensed medication from the medicine container, or combination thereof. The provisioned information can be various types of information including radio IDs, medication IDs, executable code, scheduling information, and so forth.

As another example, the RFID generator 650 can be a device that can individually provision RFID labels (or RFID chips) that are already connected to medicine containers and where the provisioning provides the information that facilitates the embodiments described above and herein. In another embodiment, the RFID generator 650 can accommodate medicine containers with or without RFID labels, such as initially determining (e.g., based on: sending a request to the medicine container to see if it responds, user input, image capture of the medicine container by image sensor 613 which may or may not include image pattern recognition to identify an RFID label being already present and/or which may or may not include a bar code reading) whether the particular medicine container has its own RFID label that should be provisioned or whether the medicine container needs a connectable RFID label (e.g., an adhesive label) which will be provisioned.

Other components not shown in FIG. 6 can be used in one or more embodiments of the subject disclosure. For instance, the communication device 600 can include a slot for adding or removing an identity module such as a Subscriber Identity Module (SIM) card or Universal Integrated Circuit Card (UICC). SIM or UICC cards can be used for identifying subscriber services, executing programs, storing subscriber data, and so on.

The terms “first,” “second,” “third,” and so forth, as used in the claims, unless otherwise clear by context, is for clarity only and doesn't otherwise indicate or imply any order in time. For instance, “a first determination,” “a second determination,” and “a third determination,” does not indicate or imply that the first determination is to be made before the second determination, or vice versa, etc.

In the subject specification, terms such as “store,” “storage,” “data store,” data storage,” “database,” and substantially any other information storage component relevant to operation and functionality of a component, refer to “memory components,” or entities embodied in a “memory” or components comprising the memory. It will be appreciated that the memory components described herein can be either volatile memory or nonvolatile memory, or can comprise both volatile and nonvolatile memory, by way of illustration, and not limitation, volatile memory, non-volatile memory, disk storage, and memory storage. Further, nonvolatile memory can be included in read only memory (ROM), programmable ROM (PROM), electrically programmable ROM (EPROM), electrically erasable ROM (EEPROM), or flash memory. Volatile memory can comprise random access memory (RAM), which acts as external cache memory. By way of illustration and not limitation, RAM is available in many forms such as synchronous RAM (SRAM), dynamic RAM (DRAM), synchronous DRAM (SDRAM), double data rate SDRAM (DDR SDRAM), enhanced SDRAM (ESDRAM), Synchlink DRAM (SLDRAM), and direct Rambus RAM (DRRAM). Additionally, the disclosed memory components of systems or methods herein are intended to comprise, without being limited to comprising, these and any other suitable types of memory.

Moreover, it will be noted that the disclosed subject matter can be practiced with other computer system configurations, comprising single-processor or multiprocessor computer systems, mini-computing devices, mainframe computers, as well as personal computers, hand-held computing devices (e.g., PDA, phone, smartphone, watch, tablet computers, netbook computers, etc.), microprocessor-based or programmable consumer or industrial electronics, and the like. The illustrated aspects can also be practiced in distributed computing environments where tasks are performed by remote processing devices that are linked through a communications network; however, some if not all aspects of the subject disclosure can be practiced on stand-alone computers. In a distributed computing environment, program modules can be located in both local and remote memory storage devices.

In one or more embodiments, information regarding use of services can be generated including services being accessed, media consumption history, user preferences, and so forth. This information can be obtained by various methods including user input, detecting types of communications (e.g., video content vs. audio content), analysis of content streams, sampling, and so forth. The generating, obtaining and/or monitoring of this information can be responsive to an authorization provided by the user. In one or more embodiments, an analysis of data or other monitoring can be subject to authorization from user(s) associated with the data, such as an opt-in, an opt-out, acknowledgement requirements, notifications, selective authorization based on types of data, and so forth.

Some of the embodiments described herein can also employ artificial intelligence (AI) to facilitate automating one or more features described herein. The embodiments (e.g., in connection with automatically identifying acquired cell sites that provide a maximum value/benefit after addition to an existing communication network) can employ various AI-based schemes for carrying out various embodiments thereof. Moreover, the classifier can be employed to determine a ranking or priority of each cell site of the acquired network. A classifier is a function that maps an input attribute vector, x=(x1, x2, x3, x4, . . . , xn), to a confidence that the input belongs to a class, that is, f(x)=confidence (class). Such classification can employ a probabilistic and/or statistical-based analysis (e.g., factoring into the analysis utilities and costs) to determine or infer an action that a user desires to be automatically performed. A support vector machine (SVM) is an example of a classifier that can be employed. The SVM operates by finding a hypersurface in the space of possible inputs, which the hypersurface attempts to split the triggering criteria from the non-triggering events. Intuitively, this makes the classification correct for testing data that is near, but not identical to training data. Other directed and undirected model classification approaches comprise, e.g., naïve Bayes, Bayesian networks, decision trees, neural networks, fuzzy logic models, and probabilistic classification models providing different patterns of independence can be employed. Classification as used herein also is inclusive of statistical regression that is utilized to develop models of priority.

As will be readily appreciated, one or more of the embodiments can employ classifiers that are explicitly trained (e.g., via a generic training data) as well as implicitly trained (e.g., via observing UE behavior, operator preferences, historical information, receiving extrinsic information). For example, SVMs can be configured via a learning or training phase within a classifier constructor and feature selection module. Thus, the classifier(s) can be used to automatically learn and perform a number of functions, including but not limited to determining according to predetermined criteria which of the acquired cell sites will benefit a maximum number of subscribers and/or which of the acquired cell sites will add minimum value to the existing communication network coverage, etc.

As used in some contexts in this application, in some embodiments, the terms “component,” “system” and the like are intended to refer to, or comprise, a computer-related entity or an entity related to an operational apparatus with one or more specific functionalities, wherein the entity can be either hardware, a combination of hardware and software, software, or software in execution. As an example, a component may be, but is not limited to being, a process running on a processor, a processor, an object, an executable, a thread of execution, computer-executable instructions, a program, and/or a computer. By way of illustration and not limitation, both an application running on a server and the server can be a component. One or more components may reside within a process and/or thread of execution and a component may be localized on one computer and/or distributed between two or more computers. In addition, these components can execute from various computer readable media having various data structures stored thereon. The components may communicate via local and/or remote processes such as in accordance with a signal having one or more data packets (e.g., data from one component interacting with another component in a local system, distributed system, and/or across a network such as the Internet with other systems via the signal). As another example, a component can be an apparatus with specific functionality provided by mechanical parts operated by electric or electronic circuitry, which is operated by a software or firmware application executed by a processor, wherein the processor can be internal or external to the apparatus and executes at least a part of the software or firmware application. As yet another example, a component can be an apparatus that provides specific functionality through electronic components without mechanical parts, the electronic components can comprise a processor therein to execute software or firmware that confers at least in part the functionality of the electronic components. While various components have been illustrated as separate components, it will be appreciated that multiple components can be implemented as a single component, or a single component can be implemented as multiple components, without departing from example embodiments.

Further, the various embodiments can be implemented as a method, apparatus or article of manufacture using standard programming and/or engineering techniques to produce software, firmware, hardware or any combination thereof to control a computer to implement the disclosed subject matter. The term “article of manufacture” as used herein is intended to encompass a computer program accessible from any computer-readable device or computer-readable storage/communications media. For example, computer readable storage media can include, but are not limited to, magnetic storage devices (e.g., hard disk, floppy disk, magnetic strips), optical disks (e.g., compact disk (CD), digital versatile disk (DVD)), smart cards, and flash memory devices (e.g., card, stick, key drive). Of course, those skilled in the art will recognize many modifications can be made to this configuration without departing from the scope or spirit of the various embodiments.

In addition, the words “example” and “exemplary” are used herein to mean serving as an instance or illustration. Any embodiment or design described herein as “example” or “exemplary” is not necessarily to be construed as preferred or advantageous over other embodiments or designs. Rather, use of the word example or exemplary is intended to present concepts in a concrete fashion. As used in this application, the term “or” is intended to mean an inclusive “or” rather than an exclusive “or”. That is, unless specified otherwise or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more” unless specified otherwise or clear from context to be directed to a singular form.

Moreover, terms such as “user equipment,” “mobile station,” “mobile,” subscriber station,” “access terminal,” “terminal,” “handset,” “mobile device” (and/or terms representing similar terminology) can refer to a wireless device utilized by a subscriber or user of a wireless communication service to receive or convey data, control, voice, video, sound, gaming or substantially any data-stream or signaling-stream. The foregoing terms are utilized interchangeably herein and with reference to the related drawings.

Furthermore, the terms “user,” “subscriber,” “customer,” “consumer” and the like are employed interchangeably throughout, unless context warrants particular distinctions among the terms. It should be appreciated that such terms can refer to human entities or automated components supported through artificial intelligence (e.g., a capacity to make inference based, at least, on complex mathematical formalisms), which can provide simulated vision, sound recognition and so forth.

As employed herein, the term “processor” can refer to substantially any computing processing unit or device comprising, but not limited to comprising, single-core processors; single-processors with software multithread execution capability; multi-core processors; multi-core processors with software multithread execution capability; multi-core processors with hardware multithread technology; parallel platforms; and parallel platforms with distributed shared memory. Additionally, a processor can refer to an integrated circuit, an application specific integrated circuit (ASIC), a digital signal processor (DSP), a field programmable gate array (FPGA), a programmable logic controller (PLC), a complex programmable logic device (CPLD), a discrete gate or transistor logic, discrete hardware components or any combination thereof designed to perform the functions described herein. Processors can exploit nano-scale architectures such as, but not limited to, molecular and quantum-dot based transistors, switches and gates, in order to optimize space usage or enhance performance of user equipment. A processor can also be implemented as a combination of computing processing units.

As used herein, terms such as “data storage,” data storage,” “database,” and substantially any other information storage component relevant to operation and functionality of a component, refer to “memory components,” or entities embodied in a “memory” or components comprising the memory. It will be appreciated that the memory components or computer-readable storage media, described herein can be either volatile memory or nonvolatile memory or can include both volatile and nonvolatile memory.

What has been described above includes mere examples of various embodiments. It is, of course, not possible to describe every conceivable combination of components or methodologies for purposes of describing these examples, but one of ordinary skill in the art can recognize that many further combinations and permutations of the present embodiments are possible. Accordingly, the embodiments disclosed and/or claimed herein are intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

In addition, a flow diagram may include a “start” and/or “continue” indication. The “start” and “continue” indications reflect that the steps presented can optionally be incorporated in or otherwise used in conjunction with other routines. In this context, “start” indicates the beginning of the first step presented and may be preceded by other activities not specifically shown. Further, the “continue” indication reflects that the steps presented may be performed multiple times and/or may be succeeded by other activities not specifically shown. Further, while a flow diagram indicates a particular ordering of steps, other orderings are likewise possible provided that the principles of causality are maintained.

As may also be used herein, the term(s) “operably coupled to”, “coupled to”, and/or “coupling” includes direct coupling between items and/or indirect coupling between items via one or more intervening items. Such items and intervening items include, but are not limited to, junctions, communication paths, components, circuit elements, circuits, functional blocks, and/or devices. As an example of indirect coupling, a signal conveyed from a first item to a second item may be modified by one or more intervening items by modifying the form, nature or format of information in a signal, while one or more elements of the information in the signal are nevertheless conveyed in a manner than can be recognized by the second item. In a further example of indirect coupling, an action in a first item can cause a reaction on the second item, as a result of actions and/or reactions in one or more intervening items.

Although specific embodiments have been illustrated and described herein, it should be appreciated that any arrangement which achieves the same or similar purpose may be substituted for the embodiments described or shown by the subject disclosure. The subject disclosure is intended to cover any and all adaptations or variations of various embodiments. Combinations of the above embodiments, and other embodiments not specifically described herein, can be used in the subject disclosure. For instance, one or more features from one or more embodiments can be combined with one or more features of one or more other embodiments. In one or more embodiments, features that are positively recited can also be negatively recited and excluded from the embodiment with or without replacement by another structural and/or functional feature. The steps or functions described with respect to the embodiments of the subject disclosure can be performed in any order. The steps or functions described with respect to the embodiments of the subject disclosure can be performed alone or in combination with other steps or functions of the subject disclosure, as well as from other embodiments or from other steps that have not been described in the subject disclosure. Further, more than or less than all of the features described with respect to an embodiment can also be utilized.

What is claimed is:

1. A device, comprising:
a processing system including a processor; and
a memory that stores executable instructions that, when executed by the processing system, facilitate performance of operations, the operations comprising:
determining a scheduled appointment at a future time for a user that is a resident of a premises in which the device is located;
receiving, over a wireless network operating in the premises, dispensing data from a wireless transmitter of a medicine container, the dispensing data indicating a time of dispensing and an identification of medication that was dispensed;
transmitting the dispensing data to a communication device associated with a healthcare provider;
determining, within a time period of the scheduled appointment, a position of the user in the premises based on communications associated with a wearable end user device that is worn by the user;
selecting a display device from among a group of display devices according to the position of the user in the premises; and
facilitating establishing a video conference with the communication device of the healthcare provider that is presented at the display device of the user.

2. The device of claim 1, wherein the operations further comprise transmitting instructions to the wearable end user device that causes the wearable end user device to present navigation information that directs movement towards the display device that was selected.

3. The device of claim 1, wherein the operations further comprise:
transmitting, to an end user device of the user, medication information indicating a reminder to take an other medication, the medication information being transmitted over a wireless network operating at least outside of the premises, the medication information indicating an identification of a wireless transceiver of an other medicine container that houses the other medication to be dispensed, wherein the end user device is a mobile phone that executes a mobile virtual assistant that is registered with a virtual healthcare assistant operating on the device located at the premises; and
receiving, over the wireless network from the end user device, dispensing data that was collected by the end user device from the wireless transceiver of the other medicine container, the dispensing data indicating a time of dispensing and an identification of the other medication that was dispensed.

4. The device of claim 1, comprising:
a Radio Frequency IDentification (RFID) generator that provisions RFID labels that are to be applied to medicine containers.

5. The device of claim 1, wherein the operations further comprise:
obtaining registration information for a group of medicine containers that house different medications;
transmitting, to the wearable end user device of the user according to a medication schedule, medication information indicating a reminder to take a particular medication;
selecting a particular medicine container that houses the particular medication from among the group of medicine containers according to the medication schedule and the registration information; and
transmitting instructions to the wearable end user device that causes the wearable end user device to present navigation information that directs movement of the user towards the particular medicine container, wherein the navigation information is generated based on localization messaging associated with the wearable end user device and an RFID label of the particular medicine container.

6. The device of claim 1, wherein the operations further comprise:
adding the dispensing data to a medical record of the user.

7. The device of claim 6, wherein the adding the dispensing data to the medical record utilizes a blockchain technology.

8. The device of claim 6, wherein the operations further comprise:
analyzing the medical record utilizing a treatment model to generate a recommendation for when to take the medication; and
transmitting instructions to the wearable end user device that causes the wearable end user device to present the recommendation.

9. The device of claim 1, wherein the operations further comprise:
detecting a reminder to take the medication;
determining, within an other time period of the reminder, an other position of the user in the premises based on other communications associated with the wearable end user device that is worn by the user;
selecting a display device from among a group of display devices according to the other position of the user in the premises; and
transmitting instructions to the display device that causes the display device to present the reminder.

10. A method, comprising:
receiving, by a processing system including a processor of an end user device of a user, medication information indicating a reminder to take a medication, the medication information being received over a wireless network from a virtual healthcare assistant operating on a customer device located at a premises where a user is a resident, the medication information indicating an identification of a wireless transceiver of a medicine container that houses the medication to be dispensed;
determining, by the processing system, a user position of the user that is remote from the premises;
determining, by the processing system, a container position of the medicine container that is remote from the premises based on communications with the wireless transceiver of the medicine container; and
presenting, by the processing system at a display of the end user device, navigation information that facilitates movement by the user towards the medicine container.

11. The method of claim 10, wherein the end user device is a mobile phone that executes a mobile virtual assistant that is registered with the virtual healthcare assistant operating on the customer device located at the premises.

12. The method of claim 11, comprising:
receiving, by the processing system, dispensing data from the wireless transceiver of the medicine container, the dispensing data indicating a time of dispensing and an identification of the medication that was dispensed; and
transmitting, by the processing system over the wireless network, the dispensing data to the virtual healthcare assistant operating on the customer device located at the premises.

13. A non-transitory machine-readable medium, comprising executable instructions that, when executed by a processing system including a processor operating on a customer device located at a premises where a user is a resident, facilitate performance of operations to provide a virtual healthcare assistant, the operations comprising:
obtaining registration information for a group of medicine containers that house different medications;
transmitting, to a wearable end user device of the user according to a medication schedule, medication information indicating a reminder to take a medication;
selecting a medicine container that houses the medication from among the group of medicine containers according to the medication schedule and the registration information; and
transmitting instructions to the wearable end user device that causes the wearable end user device to present navigation information that directs movement of the user towards the medicine container, wherein the navigation information is generated based on localization messaging associated with the wearable end user device and an RFID label of the medicine container.

14. The non-transitory machine-readable medium of claim 13, wherein the obtaining the registration information comprises receiving a wireless message from the RFID label of the medicine container.

15. The non-transitory machine-readable medium of claim 13, wherein the operations further comprise:
provisioning, by a Radio Frequency IDentification (RFID) generator of the customer device, the RFID label with medical data, the RFID label configured to be applied to the medicine container.

16. The non-transitory machine-readable medium of claim 13, wherein the operations further comprise:
transmitting, to a mobile device of the user, other medication information indicating an other reminder to take an other medication, the other medication information being transmitted over a wireless network operating at least outside of the premises, the other medication information indicating an identification of an other wireless transceiver of an other medicine container that houses the other medication to be dispensed, wherein the mobile device comprises a mobile phone that executes a mobile virtual assistant that is registered with a virtual healthcare assistant operating on the customer device located at the premises; and
receiving, over the wireless network from the mobile device, dispensing data that was collected by the mobile device from the other wireless transceiver of the other medicine container, the dispensing data indicating a time of dispensing and an identification of the other medication that was dispensed.

17. The non-transitory machine-readable medium of claim 13, wherein the operations further comprise:
receiving dispensing data from the RFID label of the medicine container, the dispensing data indicating a time of dispensing and an identification of the medication that was dispensed; and
adding the dispensing data to a medical record of the user.

18. The non-transitory machine-readable medium of claim 17, wherein the adding the dispensing data to the medical record utilizes a blockchain technology.

19. The non-transitory machine-readable medium of claim 17, wherein the operations further comprise:
analyzing the medical record utilizing a treatment model to generate a recommendation for when to take the medication; and
transmitting instructions to the wearable end user device that causes the wearable end user device to present the recommendation.

20. The non-transitory machine-readable medium of claim 13, wherein the operations further comprise:
detecting an other reminder to take the medication;
determining, within an other time period of the other reminder, an other position of the user in the premises based on other communications associated with the wearable end user device that is worn by the user;
selecting a display device from among a group of display devices according to the other position of the user in the premises; and
transmitting instructions to the display device that causes the display device to present the other reminder.
